(19) United States
(12) Patent Application Publication
(10) Pub. No.: US 2023/0048427 A1
(43) Pub. Date: Feb. 16, 2023

(54) ROTATION SCHEME FOR BACTERIAL CULTURES IN FOOD PRODUCT FERMENTATION

(71) Applicant: DSM IP ASSETS B.V., Heerlen (NL)

(72) Inventor: Pim VAN HEE, Echt (NL)

(21) Appl. No.: 17/794,346

(22) PCT Filed: Jan. 28, 2021
(86) PCT No.: PCT/EP2021/051912

§ 371 Date: Jul. 21, 2022

(30) Foreign Application Priority Data
Jan. 30, 2020 (EP) 20154679.3

(51) Int. Cl. C12N 1/20; A23L 3/3463; A23L 33/135; C12Q 1/70

(52) U.S. Cl. CPC C12N 1/20; A23L 3/3463; A23L 33/135; C12Q 1/70; C12N 2795/00011

(57) ABSTRACT

A method, system and computer program for selecting consecutive bacterial cultures for culturing a food product in a process for producing a fermented food product, the method comprising the steps of: culturing the food product with a first bacterial culture comprising at least one bacterial strain; isolating a sample during culturing with the first bacterial culture; determining at least one value indicative for a number of bacteriophages in the sample, e.g. using PCR, plaque assay, or pH; selecting a second bacterial culture for culturing the food product when the value is larger than a predetermined threshold, wherein the second bacterial culture comprises at least one bacterial strain, wherein the second bacterial culture differs from the first bacterial culture, wherein the sensitivities of the first and second bacterial culture for the bacteriophages in the sample are known and wherein the second bacterial culture is selected such as to reduce common bacteriophage sensitivities between the first and the second bacterial culture, and culturing the food product with the second bacterial culture.

### FIELD OF THE INVENTION

The invention relates to a method for determining consecutive bacterial cultures for culturing a food product. The invention further relates to a system for determining consecutive bacterial cultures for culturing a food product.

### BACKGROUND OF THE INVENTION

Fermentation in food processing is a process wherein carbohydrates are converted into organic acids or alcohol, with the use of microorganisms under anaerobic conditions. Fermentation is often performed with yeasts or bacteria as microorganisms. Almost any food product can be fermented, such as milk, olives, beans, grains, fruit such as grapes, honey, other dairy products, fish, meat and tea.

A variety of bacterial genera are used for fermentation, for example Streptococcus, Acetobacter, Bacillus, Bifidobacterium, Lactobacillus etc. Lactobacillus for example, is able to convert sugars into lactic acid, and therefore actively lowering the pH of its environment, Some Lactobacillus species can be used as starter cultures for a high variety of fermented products, such as yogurt, cheese, sauerkraut, pickles, beer, cider, kimchi, cocoa, kefir and other fermented foods.

Bacteria that are used for fermentation can be prone to bacteriophages. A bacteriophage, or just phage, is a virus that can infect and replicate within bacteria and archaea. Phages are very diverse and common, and for virtually each bacterial strain at least one phage exists that can infect said bacterial strain. Nevertheless, phages are often only able to infect and divide within a small subset of bacterial strains. The latter can be explained by either antagonistic pleiotropy, wherein adaptation to one host is advantageous but might be deleterious in another host, or when a more general mechanism of infection is chosen, efficiency is reduced. Phages can be beneficially employed as an antibacterial agent, such as in phage therapy, and have many potential applications in human medicine as well as dentistry, veterinary science, and agriculture, Because of the specificity of phages, very specific treatments can be devised.

However, bacteriophages can also cause detrimental effects. This is the case in for example, the dairy industry, wherein the bacteriophages can inhibit fermentation of diary by bacteria. Bacterial fermentation to produce other fermentation products can suffer from the same consequences. An example is the fermentation of soybean with Bacillus subtilis. Because of the specificity of bacteriophages, it is possible to change one bacterial strain for another to resolve bacteriophage infection. However, it is then necessary that the second bacterial strain is not sensitive to the same bacteriophage(s), as this would not solve the problem.

There is thus a need for bacterial rotation schemes in order to reduce the influence of bacteriophages.

### SUMMARY OF THE INVENTION

It is an object of the invention to provide for a method and a system that obviates at least one of the above mentioned drawbacks.

Additionally or alternatively, it is an object of the invention to select consecutive bacterial cultures for culturing a food product.

Additionally or alternatively, it is an object of the invention to select consecutive bacterial cultures with no common bacteriophage infection sensitivities.

Thereto, the invention provides for a method for selecting consecutive bacterial cultures for culturing a food product in a process for producing a fermented food product, the method comprising the steps of: culturing the food product with a first bacterial culture comprising at least one bacterial strain; isolating a sample during culturing with the first bacterial culture; determining at least one value indicative for a number of bacteriophages in the sample; selecting a second bacterial culture for culturing the food product when the value is larger than a predetermined threshold, wherein the second bacterial culture comprises at least one bacterial strain, wherein the second bacterial culture differs from the first bacterial culture, wherein the sensitivities of the first and second bacterial culture for the bacteriophages in the sample are known and wherein the second bacterial culture is selected such as to reduce common bacteriophage sensitivities between the first and the second bacterial culture, and culturing the food product with the second bacterial culture.

By determining at least one value indicative for a number of bacteriophages during the culturing with the first bacterial culture, one can easily determine when it is the right time to switch to a second bacterial culture, namely when a predetermined threshold is reached. At that moment, the amount of bacteriophages, and thus the concentration of bacteriophages if the total volume is known, is too high to still have an efficient acidification of the food product when applying the first bacterial culture in the fermentation process.

Another advantage of this method is that the bacteriophage count is lowered upon switching from the first bacterial culture to the second bacterial culture, due to the insensitivity of the second bacterial culture to the bacteriophages that were able to replicate on the first bacterial culture.

The predetermined critical time point is the time point at which the fermentation should be completed and the required pH is reached. To acquire this, the bacteriophage population should be kept low enough, so the bacterial culture can acidify efficiently. A threshold of a bacteriophage number has been determined in advance, for which it is known that bacterial fermentation will become too inefficient because of the large number of bacteriophages.

The term “food product”, as used herein, may refer to any substance that can be used or prepared for use as food. Food is a substance that can be eaten by an animal, such as a human, and often comprises carbohydrates, fats, proteins and/or water.

The term “culturing”, as used herein, may refer to bringing the food product in contact with a bacterial culture. The bacteria that are used in culturing are able to lower the pH of the environment. For example, in the production of cheese, bacteria will convert lactose into lactic acid. Examples of bacterial strains that are used in cheese production include Lactococcus lactis subsp. lactis or crernoris, Streptococcus salivarius subsp. thermophilus, Lactobacillus delbruckii subsp. bulgaricus, and Lactobacillus helveticus, etc.

The term “bacterial culture”, as used herein, may refer to a group of bacteria, comprising at least one strain, preferable at least two strains, more preferably at least three strains, Optionally, the first bacterial culture comprises at least a first bacterial strain and the second bacterial culture comprises at least a second bacterial strain, wherein the first bacterial strain has different bacteriophage sensitivities compared to the second bacterial strain.

The sample that is isolated during culturing with the first bacterial culture, can originate from whey, the fermenting food product, rinsing water, curd, etc. Preferably, a sample is isolated at different time points during culturing with the first bacterial culture. Preferably at least two samples are taken at least two different time points, more preferably at least three samples are taken at least three different time points.

By quantifying the bacteriophages, it would he possible to quantify the risk of infection when a bacterial culture is preceded or followed by a bacterial cultures, both sensitive to said quantified bacteriophage, The term “number of bacteriophages”, as used herein, may refer to the amount of individual bacteriophage microorganisms, regardless of the strain or type of bacteriophages. It is thus a method to quantify the bacteriophages and estimate the sensitivity of the bacterial cultures and bacterial strains of the set. The value indicative for the number of bacteriophages can be a measurement value of e.g. quantitation cycle (“C_{q} value”) in a qPCR assay. In this case, the predetermined threshold value would be the value corresponding to a predetermined maximum amount of bacteriophages, up until which acidification or fermentation is still efficient.

The terms “bacteriophage”, “phage” and the like, as used herein, may refer to a virus that is only able to infect and replicate within bacteria and archaea. Bacteriophages are composed of proteins that encapsulate a DNA or RNA genome, and often have a typical outlook of an icosahedral envelop head encapsulating the nucleic acid with a tail, made up of a sheath and a baseplate with fibers attached, The tail will attach to the bacterium or the archaea, and the nucleic acid is inserted via there, The host-microorganism is then forced to translate the DNA or RNA from the bacteriophage into bacteriophage components. After assembly, the host, e.g. the bacterium, is forced to release the phages and the bacterium is often destroyed in the process.

Bacteriophages are present everywhere, including in bulk starter cultures, Generally, bacterial cultures are rotated during the process, in the hope that the strains in the cultures are different so no common bacteriophage sensitivities are present.

The terms “sensitivity”, “bacteriophage sensitivity”, “phage sensitivity” and the like, as used herein, may refer to the ability of a bacterial culture or a bacterial strain to be infected by a specific bacteriophage. When bacterial cultures or bacterial strains are said to have a common bacteriophage sensitivity, or the like, it may refer to said bacterial cultures or bacterial strains being able to be attacked or infected by at least the same bacteriophage, but not necessarily all bacteriophages present in the bacterial cultures or bacterial strains. A bacteriophage attack or bacteriophage infection comprises the insertion of bacteriophage DNA or RNA into its host, here bacteria. Additionally, the bacteriophage DNA or RNA is replicated and translated by the bacterium, resulting in a large amount of said bacteriophage. These are then released to the environment, able to infect or attack other bacteria.

Common bacteriophage sensitivities between two bacterial cultures are often the result of the cultures comprising identical or almost identical bacterial strains. This results from the high specificity of bacteriophages for their respective host.

The term “rotation”, as used herein, may refer to a cycle wherein each bacterial culture is used once in the fermentation process before starting the process anew. This invention optimizes the order of bacterial cultures in the rotation of bacterial cultures.

In an exemplary case, the food producer chooses two bacterial cultures with each at least one bacterial strain, for which the food producer knows that no bacteriophage can infect both cultures, meaning that each of the bacterial strains between the bacterial cultures are different and have different phage sensitivities. The food producer starts to ferment a food product with the first bacterial culture, and isolates at at least one time point but preferably various time points during one production batch of the food product a sample from the food process and determines the amount of bacteriophages in the sample, by calculating or measuring a value indicative for the number of bacteriophages in the sample. In the worst case, the food producer will determine that the value is larger than a predetermined threshold, for example the value for which is known that fermentation is severely hampered and a change of bacterial culture is necessary. If the value is determined to be lower than the threshold, no change of bacterial culture is yet necessary and culturing with the first bacterial culture can continue (i.e. subsequent batches of the food product can be produced with the same bacterial culture), until the value in one of the next samples taken at later time points is larger than the predetermined threshold.

Optionally, the value indicative for the number of bacteriophages is determined at a plurality of time points during a fermentation batch of the food product, preferably at least two time points, wherein the values at the plurality of time points are used to predict a predicted value at a predetermined critical time point, wherein the step of successively culturing the food product with the second bacterial culture is initiated when the predicted value is larger than the threshold at the critical time point.

By predicting the value at a predetermined critical time point, one can already derive the bacteriophage growth speed and whether the bacteriophage population is growing too fast or not. A fast growing population, or a large population of bacteriophages is detrimental to the bacterial culture's ability to ferment the food product. Determining a value indicative for a number of bacteriophages is usually not immediately after taking a sample, but takes some time, for example with a qPCR method. If no prediction is made, it is possible that the number of bacteriophages is already too high upon taking a sample, and delays in production are not avoided.

When at least two time point values are determined, fitting can be applied, Ideally, at least three time points or more are used, in order to determine the order of the fitting graphic. A model can also be used to determine the predicted value at the predetermined critical time point. It is not necessary to wait for the determined value before isolating a following sample.

Optionally, the process for producing a fermented food product is stopped before the critical time point if the predicted value at the predetermined critical time point is equal to at least 1.2 of the predetermined threshold.

When the predicted value at the predetermined critical time point is at least 1.2 times the predetermined threshold, it can be assumed that the bacteria are not able to ferment efficiently due to the high amount of bacteriophages, and that the required pH will not be reached in the foreseeable future, It is therefore advised to finish the batch and produce the next batch with a bacterial culture with a different phage sensitivity that is unrelated. Less time would be lost, as it would otherwise only be discovered at a later time point, when it is often already too late. Optionally, another cut off value can be chosen, such as 1.1, 1.3, 1.4, 1.5 times the predetermined threshold.

Optionally, the prediction is based on an artificial intelligence model.

By using a self-learning model, a more accurate prediction can be given regarding the predicted value at the predetermined critical time point. Various artificial intelligence models are suitable for this invention.

Optionally, the value indicative for the number of bacteriophages in the sample is determined by a DNA quantification method, preferably a DNA amplification method.

As bacteriophages comprise a string of DNA or RNA, this can be used to identify or detect bacteriophages in a sample. It is required that the detection method is a fast method, wherein the detection time should be less than the time between the starting time point of the fermentation and the expected time point of the end of the fermentation. Preferably, the detection time is less than half the time between the starting time point of the fermentation and the expected time point of the end of the fermentation. More preferably, the detection time is less than a third of the time between the starting time point of the fermentation and the expected time point of the end of the fermentation.

Optionally, the value indicative for the number of bacteriophages in the sample is determined by quantitative polymerase chain reaction (qPCR).

Quantitative polymerase chain reaction (qPCR) has as advantage that it is a very quick method, thus resulting in an even faster method for detecting bacteriophage sensitivities. qPCR is also often known as real-time polymerase chain reaction (real-time PCR). Optionally, other DNA quantification methods can be used for determining the value, preferably with a DNA amplification method. PCR is widespread method for exponentially amplifying DNA sequences. This is performed by thermal cycling: when the temperature is high (94-98° C.), the DNA strain is split into two single-stranded DNA molecules due to the breaking of hydrogen bonds between the complementary bases. When the temperature is lowered to about 50-65° C., primers are able to bind to the single-stranded DNA, resulting of annealing of the primers to the single-stranded DNA. These primers are chosen specifically for which DNA sequence needs to be multiplied. In a third part of the thermal cycle, the complementary part of the single strand DNA is added by elongating the primer, by providing the mixture with a DNA polymerase, for example Thermus aquaticus (Taq) polymerase, and free deoxyribonucleotides (dNTPs), which are to be inserted, This last step is carried out at a temperature suitable for the DNA polymerase, e.g. 75-80° C. for Taq polymerase. The cycle is started anew after this last step. Usually, this is repeated for about 20-40 times.

In qPCR, the reaction is followed during the thermal cycling, for example via the addition of a non-specific fluorescent dye that is able to intercalate with any double-stranded DNA, or via the use of sequence-specific DNA probes consisting of oligonucleotides that have been labeled with a fluorescent reporter. The latter option is only detectable after hybridization with its complementary sequence.

Optionally, the value indicative of the number of bacteriophages is measured within 2 hours after sampling, more preferably within one hour after sampling, qPCR can be used to detect bacteriophage DNA present in the sample.

Optionally, a kit for detecting and quantifying bacteriophage DNA, such as a qPCR kit, comprises an instruction manual. In one of its aspects, the instruction manual comprises instructions to extract or purify the DNA from the dairy sample. In yet another aspect, the instruction manual comprises instructions to dilute the dairy sample, preferably to dilute the dairy sample with water and even more preferably to dilute the dairy sample with tap water. Preferably, the dairy sample is diluted at least 10 times, for example by mixing 5 ml of sample with water to a total volume of 50 ml or any other equivalent which results in a dilution of the dairy sample by a factor 10.

Preferably, the different components of the kit are in a lyophilized form allowing storing at ambient temperatures.

A method for detecting and quantifying phage DNA from a lactic acid bacteria infecting phage in a dairy sample may comprise the steps of:

(i) obtaining a dairy sample,

(ii) optionally, diluting the obtained dairy sample, and

(iii) testing the, optionally diluted, sample with a qPCR kit as described herein.

The dairy sample can be any of the dairy samples which are described above in the context of the kit. Typically, a dairy sample is taken at a dairy manufacturer such as a cheese or yogurt manufacturer. Preferably, a sample is taken at a cheese manufacturer. Preferably the cheese manufacturer produces cheese on large scale, i.e. a manufacturer which produces at least 3000 kg cheese per year. Or alternatively, a sample is taken from a batch or fermentation vat or fermentation vessel comprising at least 50 L of material. Yet another source of the sample is a sample from a(n) (original) pack size of at least 10 kg of powder, for example whey powder.

Preferably, the method for detecting and quantifying phage DNA from a lactic acid bacteria infecting phage in a dairy sample is performed at the dairy manufacturer, i.e. the sample does not to be transported to a test lab outside of the dairy factory,

Preferred samples taken in step (i) are samples from whey, bulk starter media, bulk starter cultures, milk, acidified milk, whey powder, rinse water, a swab from dairy processes, cheese or a fermented dairy product.

The obtained sample can be tested as such, for example rinse water can be tested as such and does not need a dilution step. Also after dissolving whey powder in water at an appropriate concentration, the whey powder sample can be tested as such and does not need a dilution step. Also after processing a swab sample in water, the sample can be tested as such and does not need a dilution step.

Other obtained samples need to be diluted, such as whey, milk, acidified milk, a fermented dairy product or bulk starter broth or media.

In optional step (ii) the sample is preferably diluted with water such as tap water, distilled water, double-distilled water or molecular grade water (e.g. MilliQ), Preferably, the dairy sample is diluted with tap water, Buffers which are qPCR compatible can also be used as a means for diluting the dairy sample,

Preferably, the dairy sample is (if at all) diluted at least ten-fold, meaning that x ml sample is diluted such as to end at x0 ml of diluted sample (for example 5 ml sample being diluted with help of 45 ml water to a total volume of 50 ml). Such a dilution can easily be performed by the factory worker by using a scoop to hold 5 mL and put it into a tube which has a visible mark at 50 ml allowing an easy dilution step. Another option would be that the factory worker uses a micropipette, which pipettes a fixed volume, or a pastette to pipette 20 microliters of a dairy sample to a 5 mL tube containing 2.48 mL of water, in this way diluting The sample 125-fold.

Subsequently, 20 to 100 microliters of the diluted sample is transferred to a reaction vessel with freeze-dried “phage test reaction mixture” with a pastette or micropipette. The to be tested reaction vessels are transferred to a suitable qPCR cycler with for example a fluorescence reader such as Biorad CFX system or a suitable mobile device such as three9™ (Biomeme).

Preferably, a method as described herein makes use of a portable device in step (iii). More preferably the portable device is a portable device with a display on which the phage risk level is displayed. I.e. the portable device translates the results of the qPCR analysis into an advice for the dairy manufacturer, for example to use another rotation of lactic acid bacteria.

Preferably, a method as described herein does not comprise a DNA extraction step or a DNA purification step.

A method as described herein is a fast method meaning that the detection and quantification is finished within a couple of hours. Preferably, step (iii) of said method is completed within 2 hours, preferably within 90 minutes and more preferably within 60 minutes.

Optionally, the second bacterial culture is determined to be compatible with the first bacterial culture for subsequently culturing the food product when no common bacteriophage sensitivity exists between the at least one bacterial strain of the first bacterial culture and the at least one bacterial strain of the second bacterial culture,

Ideally, consecutive bacterial cultures have no common bacteriophage infection sensitivities, meaning that no bacteriophage can grow on and/or infect both bacterial cultures. To obtain this, none of the bacterial strains can be identical between the first and the second bacterial culture, as bacteriophages will be able to target these as well. This would result in further bacteriophage quantity growth, furthermore slow down the bacterial growth and its capacity to acidify its environment. The efficiency of the fermentation will thus be lowered or even haltered if common bacteriophage sensitivities are ubiquitous or even present.

Optionally, the sensitivity to bacteriophages for the first and second bacterial cultures is determined by accessing a database,

The main advantage of using a database is that no further experiments are necessary to determine bacteriophage sensitivities. Such databases are often drawn up by providers of bacterial cultures and comprise information regarding bacteriophage sensitivities, and compatibility between bacterial cultures.

Optionally, a compatibility matrix for a plurality of bacterial strains is accessed, wherein the compatibility matrix indicates compatibility between at least said first and second bacterial cultures, wherein the compatibility is based on bacteriophage sensitivity of the at least one bacterial strains in the first and second bacterial cultures.

If information regarding bacteriophage sensitivity is available for more than two bacterial cultures or bacterial strains, an optimal combination can be chosen wherein no common sensitivities are present. When a bacterial culture comprises more than one bacterial strain, these bacterial strains are present in a certain proportion. To minimize common bacteriophage sensitivities, and thus optimize the process, common bacteriophage sensitivities of bacterial strains in high proportion in the bacterial culture should be avoided in consecutive bacterial cultures, When it is not possible to remove all common bacteriophage sensitivities in consecutive bacterial cultures, common bacteriophage sensitivities of bacterial strains in low proportion in consecutive bacterial cultures can be chosen. A model can be used to solve this optimization problem.

Optionally, the value indicative for the number of bacteriophages in the sample is determined by a phage plaque assay.

The phage plaque assay is an easy and straightforward method to detect virulent bacteriophages, meaning phages that damage their host cell, here a bacterium. A drawback of this method is the duration of the assay, as bacteria and bacteriophages need time to grow in a petri dish or another suitable container.

Presence of bacteriophages is detected as plaques are formed that are visible with the naked eye, plaques being spots on the surface where no bacteria are growing, as the bacteriophage has infected an initial bacterium, and has spread to bacteria surrounding said initial bacterium, By testing the sample at various dilutions of bacteriophages, the initial concentration of bacteriophages can be determined via i.e. plaque forming units (PFU) in a sample.

Optionally, the value indicative for the number of bacteriophages in the sample is determined by pH measurements during culturing of the food product with the first bacterial culture.

Acidification efficiency of a bacteria culture largely depends on how many bacteria are actively fermenting. Upon bacteriophage infection, bacteria are killed and are therefore no longer able to lower the pH. Therefore, monitoring the value of the pH is a good indication of the number of bacteriophages, as bacteriophages actively slow down the lowering of pH.

Optionally, culturing the food product with the second bacterial culture is initiated when a predetermined threshold pH value during culturing of the food product with the first bacterial culture is not reached within a predetermined period of time after starting culturing the food product with the first bacterial culture.

As the resulting pH is the most important goal for fermentation of a food product, it is a useful parameter to evaluate the fermentation process. If the pH is not lowering fast enough, it could be an indication of too many bacteriophages in the bacterial culture. Whenever it becomes clear that the predetermined pH will not be reached in a predetermined amount of time, starting from the start of the fermentation, the first bacterial culture can be switched out for the second bacterial culture. The second bacterial culture, which is not sensitive to the same bacteriophages, will he able to acidify the food product further.

Optionally, determining the value indicative for the number of bacteriophages is performed by detecting and/or identifying bacteriophages in the isolated sample.

Multiple methods exist to determine a value that represents the amount of bacteriophages in a certain sample, such as phage plaque assay, or qPCR. The value that is obtained can often be recalculated via a calibration curve to the true number of bacteriophages that were present in the sample. Calibration can be performed from known concentrations of bacteriophages, such as determined via a phage plaque assay.

Optionally, bacterial cultures are identified by means of a readable code provided in and/or on a packaging of said bacterial cultures.

Providers of bacterial cultures can provide the bacterial cultures in a powdered or frozen form, being packaged for easy recognition and storage. On the packaging, a readable code such as a barcode or a quick response code (QR code) can be placed, This allows for a fast recognition of the bacterial culture and suitable compatibility with other bacterial cultures can be provided upon reading said readable code, with e.g. a computer application, such as an application for a smartphone.

Optionally, the fermented food product is a dairy product, preferably a cheese or a yoghurt. Optionally the initial food product that is cultured is a dairy product, such as milk.

Bacteriophages are a large hazard in the fermentation of milk, as they can infect the bacteria that are used for the acidification of milk in the production of fermented milk products, such as yogurt, sour milk, quark, cream cheese, soft cheese, semi-soft cheese, semi-hard cheese, hard cheese, soured cream, cultured butter, sour cream, creme fraiche, mascarpone, sour milk cheese, buttermilk, schmand or smetana, blue vein cheese, etc.

This acidification can be performed with a wide range of bacteria, preferably lactic acid bacteria, such as Lactococcus spp. including Lactococcus lactis ssp. lactis and Lactococcus lactis ssp. cremoris, Streptococcus spp. such as Streptococcus salivarius ssp. thermophiles, Lactobacillus spp. such as Lactobacillus heiveticus, Lactobacillus delbruckii subsp. delbrueckii, subsp. lactis and subsp, bulgaricus, Lactobacillus plantarum, Lactobacillus sanfranciscensis, Lactobacillus paracasel subsp. paracasei, Lactobacillus curvatus, Lactobacillus rhaninosus, Lactobacillus casei, and Lactobacillus helveticus, Propionibacter spp. such as Propionibacter shermani, Leuconostoc spp. such as Leuconostoc mesenteroides, etc.

Milk can be from an animal source, e.g, cow, goat, sheep, buffalo, etc, Additionally, milk can also have a non-dairy source, such as plant milk. Examples of plant milk include almond milk, coconut milk, rice milk, soy milk, etc.

Optionally, the first bacterial culture comprises at least a first bacterial strain and the second bacterial culture comprises at least a second bacterial strain, wherein the first bacterial strain has different bacteriophage sensitivities compared to the second bacterial strain.

Optionally, the invention further comprises isolating a sample during culturing of the food product with the second bacterial culture, determining at least one value indicative for a number of bacteriophages in the sample; selecting a third bacterial culture for culturing the food product when the value is larger than a predetermined threshold, wherein the third bacterial culture comprises at least one bacterial strain, wherein the third bacterial culture differs from the first and from the second bacterial culture, wherein the sensitivities of the first, second and third bacterial cultures for the bacteriophages in the second sample are known and wherein the third bacterial culture is selected such as to reduce common bacteriophage sensitivities between the first and second bacterial culture, and said third bacterial culture, and culturing the food product with the third bacterial culture.

By including this third step, a third bacterial culture is added to the bacterial rotation scheme. Optionally, the same process can be repeated with even more bacterial cultures. The main advantage of this is to create a more phage robust process, as more variation exists in bacteriophage sensitivity if the bacterial cultures do not have any common bacteriophage sensitivities in successive bacterial cultures. However, it is also important to take into account the continuity of the characteristics of the resulting fermented food product, which should be the same for each bacterial culture that is used in the process. Therefore, an optimal amount of bacterial cultures is three, four or five bacterial cultures. In this case, the characteristics of the resulting fermented food product should be conserved, while still providing a robust bacterial rotation scheme, as opposed to only one or two bacterial cultures.

According to an aspect, the invention provides for a system for selecting consecutive bacterial strains for culturing a food product in a process for producing a fermented food product, the system including: a controller; means for culturing the food product, and an analysis unit for monitoring a value indicative for the number of bacteriophages in a sample of the process for producing a fermented food product; and wherein the controller is configured to operate the system to perform the steps of: culturing the food product with a first bacterial culture comprising at least one bacterial strain; determining, by means of the analysis unit, a value indicative for a number of bacteriophages in the sample of the process for producing a fermented food product; selecting a second bacterial culture for culturing the food product when the value is larger than a predetermined threshold, the second bacterial culture comprising at least one bacterial strain and differing from the first bacterial culture, the sensitivities of the first and second bacterial culture for the bacteriophages in the sample are known and wherein the second bacterial culture is selected such as to reduce common bacteriophage sensitivities between the first and the second bacterial culture; and subsequently culturing the food product with the second bacterial culture.

According to an aspect, the invention provides for a computer program product configured to be run on a machine for selecting consecutive bacterial cultures for culturing a food product in a process for producing a fermented food product, the computer program product being configured to: receiving a value indicative for the number of bacteriophages in a sample of the process for producing a fermented food product during culturing the food product with a first bacterial culture including at least one bacterial strain; selecting a second bacterial culture for subsequently culturing the food product, when the value is larger than a predetermined threshold, the second bacterial culture comprising at least one bacterial strain and differing from the first bacterial culture, the sensitivities of the first and second bacterial culture for the bacteriophages in the sample are known and wherein the second bacterial culture is selected such as to reduce common bacteriophage sensitivities between the first and the second bacterial culture.

It will be appreciated that any of the aspects, features and options described in view of the method apply equally to the system and the described computer program. It will also be clear that any one or more of the above aspects, features and options can be combined.

### BRIEF DESCRIPTION OF THE DRAWINGS

The invention will further be elucidated on the basis of exemplary embodiments which are represented in a drawing. The exemplary embodiments are given by way of non-limitative illustration; It is noted that the figures are only schematic representations of embodiments of the invention that are given by way of non-limiting example.

In the drawings:

FIG. 1 shows a schematic diagram of an embodiment of a method for selecting consecutive bacterial cultures for culturing a food product;

FIG. 2A shows a schematic diagram of acidification and the influence of a phage infection;

FIG. 2B shows a schematic diagram of an embodiment of a method for rotating bacterial cultures in function of phage concentration;

FIG. 3 shows a schematic diagram of an embodiment of a compatibility matrix of bacterial cultures regarding bacteriophage sensitivity; and

FIG. 4 shows a schematic diagram of a method of the invention;

### DETAILED DESCRIPTION OF THE INVENTION

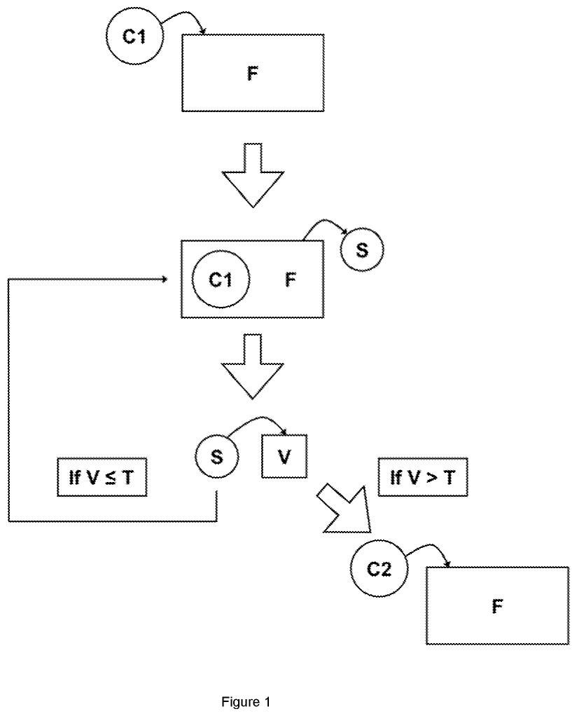

FIG. 1 shows a schematic diagram of an embodiment of a method for selecting consecutive bacterial cultures for culturing a food product. In a first step, the food F is cultured with a first bacterial culture C1 comprising at least one bacterial strain. In a second step, a sample S is isolated from the first bacterial culture during culturing. In a third step, at least one value V indicative for a number of bacteriophages in the sample S is determined. When the value is larger than a predetermined threshold T, the next batch of the food F is cultured with a second bacterial culture C2. The second bacterial culture comprises at least one bacterial strain, differs from the first bacterial culture C1 and the sensitivities of the first bacterial culture (C1) and second bacterial culture (C2) for the bacteriophages in the sample S are known and wherein the second bacterial culture C2 is selected such as to reduce common bacteriophage sensitivities between the first bacterial culture C1 and the second bacterial culture C2.

Optionally, when the value V is not larger than the predetermined threshold T, culturing is continued with the first bacterial culture C1, until a value V is determined that is larger than threshold T.

The sample S is a process sample, that is taken from anything that came into contact with said bacterial culture, e.g. rinsing water, whey, fermented food product, curd, etc.

Preferably, the invention comprises at least two values V, more preferably three values V, each taken at a different time point after the starting of the culturing.

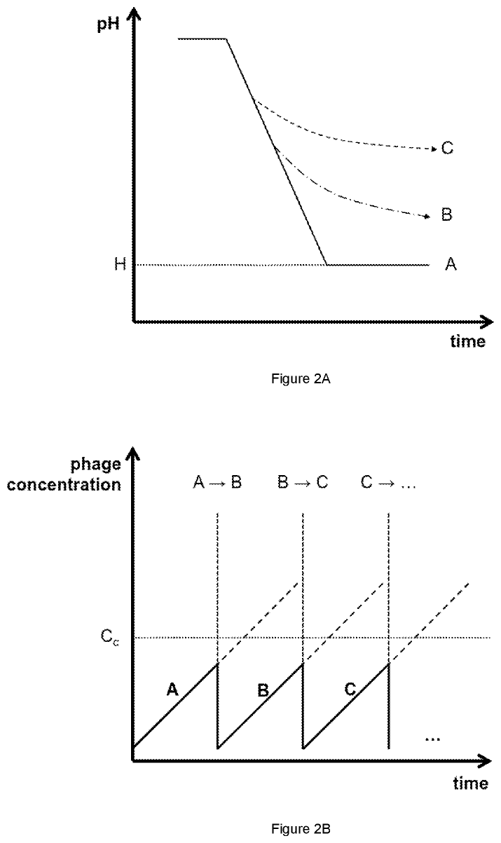

FIG. 2A shows a schematic diagram of acidification and the influence of a phage infection. In a graphical view of the pH in function of the time, an ideal situation would be outcome A. Threshold H is the ideal pH after acidification, which has been reached in situation A, wherein no influence of bacteriophages is detected. In case of a moderate phage infection, the acidification, thus lowering of the pH, will be slowed down and a longer time is necessary to reach threshold pH. This is depicted by outcome B, Lastly, in case of a severe phage infection, outcome C is reached, The acidification is compromised to such a degree, that reaching the threshold pH is unlikely or will take a long time.

FIG. 2B shows a schematic diagram of an embodiment of a method for rotating bacterial cultures in function of phage concentration. When bacterial culture A is used, the concentration of bacteriophages against bacterial culture A will increase, either linear, as depicted in FIG. 2B, or non-linear, After a specific time, or a specific pH threshold is reached, bacterial culture A will be replaced by bacterial culture B, as depicted by the vertical dotted line. If bacterial culture B is compatible with bacterial culture A, thus no common bacteriophage sensitivities are present, the bacteriophage concentration is reduced to a basal level, at which point different bacteriophages can start growing, which can infect bacterial strains from bacterial culture B. In the case where bacterial culture is incompatible with bacterial culture A, thus common bacteriophage sensitivities exist, the leftmost diagonal dotted line may appear. In this case, the same bacteriophages will be able to multiply further and will reach a critical concentration C_{c}, wherein the acidification with bacterial culture B will no longer be able to reach the desired pH. The phage concentration growth is here depicted as linear, but may also be non-linear. In the case wherein bacterial culture A and B are compatible, the process can be continued and repeated with bacterial culture C, etc.

Bacteriophages are present everywhere, including bulk starter cultures, Generally, bacterial cultures will be rotated during the process, in the hope that the strains in the cultures are different so no common bacteriophage sensitivities are present.

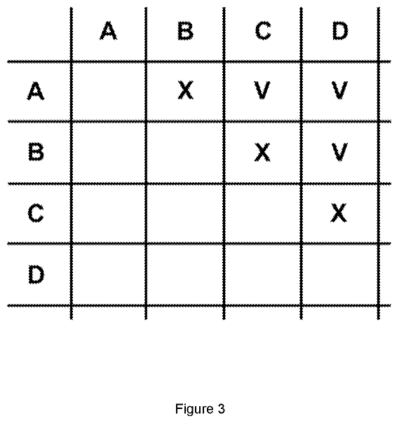

FIG. 3 shows a schematic diagram of an embodiment of a compatibility matrix of bacterial cultures regarding bacteriophage sensitivity. A “V” signifies bacteriophage compatibility, thus no common bacteriophage sensitivities, an “X” signifies bacteriophage incompatibility, thus having common bacteriophage sensitivities. In this example, A and C are compatible and can thus be successive in an adapted order. A and B are incompatible, and it should thus be avoided to use said bacterial cultures successively. It should be understood that while FIG. 3 shows 4 bacterial cultures, the compatibility matrix can comprise any number of cultures. Additionally, it is not a requirement that the matrix is completely filled as data may be missing. Preferably, the compatibility matrix is filled out completely.

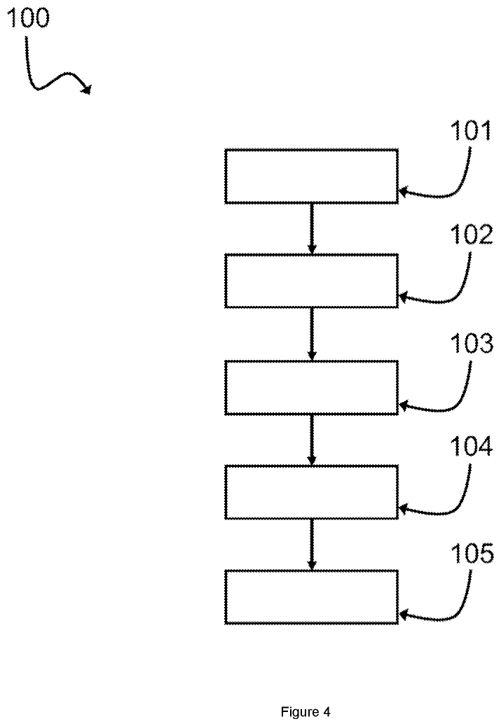

FIG. 4 shows a schematic diagram of a method 100 for selecting consecutive bacterial cultures for culturing a food product in a process for producing a fermented food product. In some examples, part of the method is a computer implemented method configured to be run on a machine. In a first step 101, the food product is cultured with a first bacterial culture comprising at least one bacterial strain. In a second step 102, a sample is isolated during culturing with the first bacterial culture. In a third step 103, at least one value is determined indicative for a number of bacteriophages in the sample. In a fourth step 104, a second bacterial culture is selected for culturing the food product when the value is larger than a predetermined threshold, wherein the second bacterial culture comprises at least one bacterial strain, wherein the second bacterial culture differs from the first bacterial culture, wherein the sensitivities of the first and second bacterial culture for the bacteriophages in the sample are known and wherein the second bacterial culture is selected such as to reduce common bacteriophage sensitivities between the first and the second bacterial culture. In a fifth step 105, the food product is cultured with the second bacterial culture.

It will be appreciated that the method may include computer implemented steps. All above mentioned steps can be computer implemented steps. Embodiments may comprise computer apparatus, wherein processes performed in computer apparatus. The invention also extends to computer programs, particularly computer programs on or in a carrier, adapted for putting the invention into practice. The program may be in the form of source or object code or in any other form suitable for use in the implementation of the processes according to the invention. The carrier may be any entity or device capable of carrying the program, For example, the carrier may comprise a storage medium, such as a ROM, for example a semiconductor ROM or hard disk. Further, the carrier may be a transmissible carrier such as an electrical or optical signal which may be conveyed via electrical or optical cable or by radio or other means, e.g. via the internet or cloud.

Some embodiments may be implemented, for example, using a machine or tangible computer-readable medium or article which may store an instruction or a set of instructions that, if executed by a machine, may cause the machine to perform a method and/or operations in accordance with the embodiments,

Various embodiments may be implemented using hardware elements, software elements, or a combination of both, Examples of hardware elements may include processors, microprocessors, circuits, application specific integrated circuits (ASIC), programmable logic devices (PLD), digital signal processors (DSP), field programmable gate array (FPGA), logic gates, registers, semiconductor device, microchips, chip sets, et cetera. Examples of software may include software components, programs, applications, computer programs, application programs, system programs, machine programs, operating system software, mobile apps, middleware, firmware, software modules, routines, subroutines, functions, computer implemented methods, procedures, software interfaces, application program interfaces (API), methods, instruction sets, computing code, computer code, et cetera.

Herein, the invention is described with reference to specific examples of embodiments of the invention. It will, however, be evident that various modifications, variations, alternatives and changes may be made therein, without departing from the essence of the invention, For the purpose of clarity and a concise description features are described herein as part of the same or separate embodiments, however, alternative embodiments having combinations of all or some of the features described in these separate embodiments are also envisaged and understood to fall within the framework of the invention as outlined by the claims. The specifications, figures and examples are, accordingly, to be regarded in an illustrative sense rather than in a restrictive sense. The invention is intended to embrace all alternatives, modifications and variations which fall within the spirit and scope of the appended claims. Further, many of the elements that are described are functional entities that may be implemented as discrete or distributed components or in conjunction with other components, in any suitable combination and location.

In the claims, any reference signs placed between parentheses shall not be construed as limiting the claim. The word ‘comprising’ does not exclude the presence of other features or steps than those listed in a claim. Furthermore, the words ‘a’ and ‘an’ shall not be construed as limited to ‘only one’, but instead are used to mean ‘at least one’, and do not exclude a plurality. The mere fact that certain measures are recited in mutually different claims does not indicate that a combination of these measures cannot be used to an advantage.

1. A method for selecting consecutive bacterial cultures for culturing a food product in a process for producing a fermented food product, the method comprising:
culturing the food product with a first bacterial culture comprising at least one bacterial strain;
isolating a sample during culturing with the first bacterial culture;
determining at least one value indicative for a number of bacteriophages in the sample;
selecting a second bacterial culture for culturing the food product when the value is larger than a predetermined threshold,
wherein the second bacterial culture comprises at least one bacterial strain,
wherein the second bacterial culture differs from the first bacterial culture,
wherein sensitivities of the first and second bacterial culture for the bacteriophages in the sample are known and wherein the second bacterial culture is selected optionally to reduce common bacteriophage sensitivities between the first and the second bacterial culture, and
culturing the food product with the second bacterial culture.

2. The method according to claim 1, comprising determining the value indicative for the number of bacteriophages at a plurality of time points, optionally at least two time points, wherein the values at the plurality of time points are used to predict a predicted value at a predetermined critical time point, wherein the successively culturing the food product with the second bacterial culture is initiated when the predicted value is larger than the threshold at the critical time point.

3. The method according to claim 2, wherein the prediction is based on an artificial intelligence model.

4. The method according to claim 1, wherein determining the value indicative for the number of bacteriophages is performed by detecting and/or identifying bacteriophages in the isolated sample.

5. The method according to claim 1, wherein the value indicative for the number of bacteriophages in the sample is determined by a DNA quantification method, optionally a DNA amplification method.

6. The method according to claim 5, wherein the value indicative for the number of bacteriophages in the sample is determined by quantitative polymerase chain reaction (qPCR).

7. The method according to claim 1, wherein the value indicative for the number of bacteriophages in the sample is determined by a phage plaque assay.

8. The method according to claim 1, wherein the value indicative for the number of bacteriophages in the sample is determined by pH measurements during culturing of the food product with the first bacterial culture.

9. The method according to claim 1, wherein the second bacterial culture is determined to be compatible with the first bacterial culture for subsequently culturing the food product when no common bacteriophage sensitivity exists between the at least one bacterial strain of the first bacterial culture and the at least one bacterial strain of the second bacterial culture.

10. The method according to claim 1, wherein the sensitivity to bacteriophages for the first and second bacterial cultures is determined by accessing a database.

11. The method according to claim 1, further including accessing a compatibility matrix for a plurality of bacterial strains, wherein the compatibility matrix indicates compatibility between at least said first and second bacterial cultures, wherein the compatibility is based on bacteriophage sensitivity of the at least one bacterial strain in the first and second bacterial cultures.

12. The method according to claim 8, wherein culturing the food product with the second bacterial culture is initiated when a predetermined threshold pH value during culturing of the food product with the first bacterial culture is not reached within a predetermined period of time after starting culturing the food product with the first bacterial culture.

13. The method according to claim 1, wherein bacterial cultures are identified by means of a readable code provided in and/or on a packaging of said bacterial cultures.

14. The method according to claim 1, wherein the first bacterial culture comprises at least a first bacterial strain and the second bacterial culture comprises at least a second bacterial strain, wherein the first bacterial strain has different bacteriophage sensitivities compared to the second bacterial strain.

15. The method according to claim 1, further comprising:
isolating a second sample during culturing of the food product with the second bacterial culture,
determining at least one value indicative for a number of bacteriophages in the second sample;
selecting a third bacterial culture for culturing the food product when the value is larger than a predetermined threshold,
wherein the third bacterial culture comprises at least one bacterial strain,
wherein the third bacterial culture differs from the first and from the second bacterial culture,
wherein the sensitivities of the first, second and third bacterial cultures for the bacteriophages in the second sample are known and wherein the third bacterial culture is selected optionally to reduce common bacteriophage sensitivities between the first and second bacterial culture, and said third bacterial culture, and
culturing the food product with the third bacterial culture.

16. A system for selecting consecutive bacterial strains for culturing a food product in a process for producing a fermented food product, the system comprising:
a controller;
means for culturing the food product, and
an analysis unit for monitoring a value indicative for the number of bacteriophages in a sample of the process for producing a fermented food product; and
wherein the controller is configured to operate the system to perform:
culturing the food product with a first bacterial culture comprising at least one bacterial strain;
determining, by means of the analysis unit, a value indicative for the number of bacteriophages in the sample of the process for producing a fermented food product;
selecting a second bacterial culture for culturing the food product when the value is larger than a predetermined threshold, the second bacterial culture comprising at least one bacterial strain and differing from the first bacterial culture, sensitivities of the first and second bacterial culture for the bacteriophages in the sample are known and wherein the second bacterial culture is selected optionally to reduce common bacteriophage sensitivities between the first and the second bacterial culture; and
subsequently culturing the food product with the second bacterial culture.

17. A computer program product configured to run on a machine for selecting consecutive bacterial cultures for culturing a food product in a process for producing a fermented food product, the computer program product being configured to:
receive a value indicative for the number of bacteriophages in a sample of the process for producing a fermented food product during culturing the food product with a first bacterial culture including at least one bacterial strain;
select a second bacterial culture for subsequently culturing the food product, when the value is larger than a predetermined threshold, the second bacterial culture comprising at least one bacterial strain and differing from the first bacterial culture, sensitivities of the first and second bacterial culture for the bacteriophages in the sample are known and wherein the second bacterial culture is selected optionally to reduce common bacteriophage sensitivities between the first and the second bacterial culture.
